(19) United States
(12) Patent Application Publication
(10) Pub. No.: US 2010/0274223 A1
(43) Pub. Date: Oct. 28, 2010

(54) REPETITIVE ENTRY CONDUIT FOR BLOOD VESSELS

(76) Inventors: George P. Teitelbaum, Santa Monica, CA (US); Samuel M. Shaolian, Newport Beach, CA (US); Scott L. Pool, Laguna Hills, CA (US)

Correspondence Address: STOEL RIVES LLP - SLC, 201 SOUTH MAIN STREET, SUITE 1100, ONE UTAH CENTER, SALT LAKE CITY, UT, 84111, US

(21) Appl. No.: 12/765,158

(22) Filed: Apr. 22, 2010

Related U.S. Application Data
(60) Provisional application No. 61/171,512, filed on Apr. 22, 2009.

(51) Int. Cl. A61M 5/32

(52) U.S. Cl. 604/507; 604/175

(57) ABSTRACT

A subcutaneous needle conduit attaches directly to a blood vessel or other biological boundary structure. The subcutaneous needle conduit is tapered such that a proximal end is wider than a distal end. A body of the subcutaneous needle conduit guides the tip of a needle or other canula from the proximal end to the distal end. The subcutaneous needle conduit may be funnel-shaped. An elongated funnel shape may be used to selectively provide access to a plurality of desired access sites along an axis of a blood vessel. Other shapes, such as sluice-shaped, may also be used. The subcutaneous needle conduit may be located beneath the skin surface using, for example, tactile sensation, magnetism, metal detection, detection of a signal emitted from a minute transponder, detection of light emission, or through other detection methods.

### RELATED APPLICATION

This application claims the benefit under 35 U.S.C. §119(e) of U.S. Provisional Application No. 61/171,512, filed Apr. 22, 2009, which is hereby incorporated by reference herein in its entirety.

### TECHNICAL FIELD

This disclosure relates to subcutaneous vascular access ports.

### BACKGROUND INFORMATION

Repetitive vascular access is used for treatments such as prolonged intravenous chemotherapy protocols and venous hemodialysis.

Among the patients needing repeated vascular access are chemotherapy patients. A large number of chemotherapeutic agents are infused intravenously over multiple cycles during the treatment of a wide variety of neoplasms. Because many of these agents can cause pain and vessel thrombosis and sclerosis, these chemotherapeutic agents are generally infused into a larger central vein by means of a peripherally inserted central catheter or “PIC line” (e.g., a size 4 French (F) or lower diameter catheter may be inserted, usually, into a basilic or cephalic vein of the upper extremity), the distal tip of which is advanced into a central vein such as the superior vena cava. However, the PIC line can occlude and can cause phlebitis with propagation of clot centrally that may require long-term anticoagulation therapy as well as removal of the PIC line. In addition, because the infusion port of a PIC lines is outside the skin, it is possible for infection to track along the course of the catheter.

Chemotherapeutic infusions can also be accomplished through infusion port catheters where injectable infusion ports are implanted subcutaneously, usually in the upper chest region. The distal catheters of infusion port catheter devices are usually inserted into the superior vena cava via a puncture site within the subclavian or jugular veins. Infusion port catheters are also subject to occlusion and phlebitis. Also, stenoses (narrowing) can develop at the catheter insertion site within the subclavian or jugular vein.

Dialysis patients may also need repeated vascular access. Currently, more than 350,000 Americans are undergoing hemodialysis approximately three times a week for chronic renal failure. Although this is often accomplished using a surgically created upper extremity arteriovenous (AV) fistula (a polytetrafluoroethylene (PTFE) graft connecting an artery and a vein in the forearm or upper arm which has replaced the Scribner shunt), at times peritoneal dialysis or venous hemodialysis are used. Problems associated with AV fistula hemodialysis include frequent shunt thrombosis that requires a semi-emergent thrombolysis/thrombectomy+/−balloon angioplasty procedure performed by a vascular interventionist. This type of costly intervention may be required two to four times per year. These AV fistulas may also be associated with anastomotic stenoses as well as more central venous stenoses. Dialysis shunts may ultimately fail after several years of use, thus progressively limiting future options for creating a new hemodialysis access site. Peritoneal dialysis is generally less convenient than hemodialysis and entails the risk of serious or life-threatening peritonitis.

Venous hemodialysis has an advantage of minimally invasive access catheter insertion (with no open surgical procedure). However, currently its disadvantages include thrombophlebitis, thromboembolization, and entry site venous stenoses (which are significantly more difficult to treat than arterial stenoses). Once such a stenosis develops in the subclavian vein, attempts at using the ipsilateral upper extremity for the surgical creation of an AV fistula for hemodialysis are frequently unsuccessful. Typically, venous hemodialysis requires an indwelling approximately 14 F (French catheter scale, in which the diameter in millimeters can be determined by dividing the French size by three) dual lumen (one lumen for withdrawing blood and the other for reinjection of the blood returning from the hemodialysis unit) hemodialysis catheter that has its proximal ports protruding from the skin surface. This long-term surface access increases the risk of infection tracking from the skin surface along the catheter shaft and into the deep perivenous tissues and even into the intravascular space (an AV hemodialysis fistula is entirely subcutaneous).

### SUMMARY

In certain embodiments, a subcutaneous needle conduit attaches directly to a blood vessel or other biological boundary structure. The subcutaneous needle conduit is tapered such that a proximal end is wider than a distal end. A body of the subcutaneous needle conduit guides the tip of a needle or other canula from the proximal end to the distal end. The subcutaneous needle conduit may be funnel-shaped. An elongated funnel shape may be used to selectively provide access to a plurality of desired access sites along an axis of a blood vessel. Other shapes, such as sluice-shaped, may also be used. The subcutaneous needle conduit may be located beneath the skin surface using, for example, tactile sensation, magnetism, metal detection, detection of a signal emitted from a minute transponder, detection of light emission, or through other detection methods.

In one embodiment, a subcutaneous conduit for implanting in a patient to allow repeated access to a blood vessel includes a tapered guide segment for guiding a needle through subcutaneous tissue to the blood vessel, a proximal opening for receiving the needle into the guide segment, and a distal opening for passing a tip of the needle out of the guide segment into the blood vessel. The distal opening is sized and configured to be attached directly to a surface of the blood vessel. The proximal opening is wider than the distal opening such that a width of the guide segment tapers from the proximal opening to the distal opening.

In one embodiment, a method for repeatedly accessing a blood vessel includes making an incision in a patient's skin at an incision location, and excising a pouch in subcutaneous tissue for receiving a funnel-shaped conduit including a proximal opening, a distal opening, and a tapered guide segment extending between the proximal opening and the distal opening. The method further includes inserting the funnel-shaped conduit into the pouch, securing the distal opening of the funnel-shaped conduit directly to a surface of the blood vessel, securing the proximal opening to the subcutaneous tissue, and repairing the incision. After a healing period, a needle may be inserted through the patient's skin so as to enter the guide segment through the proximal opening and advance a tip of the needle through the distal opening into the blood vessel.

In one embodiment, a subcutaneous conduit includes an eyelet segment adapted to engage against the surface of a biological boundary structure. The subcutaneous conduit also includes a guide segment that includes a puncture-resistant surface shaped to narrow such that a needle can first engage a larger segment and be guided by a shaped, narrowing surface towards the eyelet segment. The subcutaneous conduit may also include tabs, located near the eyelet segment, adapted to engage a ligating mechanism for attaching the needle conduit to the biological boundary structure.

In one embodiment, a subcutaneous conduit includes an eyelet segment adapted to engage against the surface of a biological boundary structure. The subcutaneous conduit also includes a guide segment that includes means for guiding the needle towards the eyelet segment. The subcutaneous conduit may also include means, located near the eyelet segment, adapted to engage a ligating mechanism for attaching the needle conduit to the biological boundary structure.

In one embodiment, a system for accessing a biological boundary structure at a desired entry site within a mammalian body includes one or more metal detector coils within a coil housing. The one or more metal detector coils are configured to detect a metallic implant within the mammalian body. The system also includes a guide canula attached to the coil housing at an angle such that placement of the one or more metal detector coils over the metallic implant aligns an opening through the guide canula with desired entry site.

Additional aspects and advantages will be apparent from the following detailed description of preferred embodiments, which proceeds with reference to the accompanying drawings.

### BRIEF DESCRIPTION OF THE DRAWINGS

FIG. 1 is a perspective view of a funnel-shaped subcutaneous conduit shown as affixed to a blood vessel according to one embodiment.

FIG. 2 is a cross-section view of the funnel-shaped subcutaneous conduit shown in FIG. 1 implanted in a patient according to one embodiment.

FIG. 3A is a cross-section view of a sheathed needle accessing a blood vessel through the funnel-shaped subcutaneous conduit according to one embodiment.

FIG. 3B is a cross-section of the funnel-shaped subcutaneous conduit in a coapted state according to one embodiment.

FIGS. 4A, 4B, 4C, 4D, 4E, and 4F are cross-section views illustrating a method for inserting the funnel-shaped subcutaneous conduit into a patient according to one embodiment.

FIGS. 5A and 5B are enlarged cross-section views of a hypotube positioned adjacent to a tab of the funnel-shaped subcutaneous conduit according to one embodiment.

FIG. 6 is a perspective view of a sluice-shaped or “dust pan” subcutaneous conduit according to one embodiment.

FIGS. 7A, 7B, 7C, 7D, and 7E schematically illustrate an elongated funnel-shaped subcutaneous conduit according to certain embodiments.

FIGS. 8A, 8B, and 8C schematically illustrate a metal detection system for accessing a blood vessel according to one embodiment.

### DETAILED DESCRIPTION

Intravenous chemotherapy infusion and venous hemodialysis may be significantly improved if one could avoid the use of in-dwelling catheters to accomplish these techniques. If one could rapidly and safely access a larger central vein, while minimizing trauma to this vessel, and could reliably access such a vessel repeatedly over the course of months to years, one could avoid or limit the problems of venous entry site stenosis, phlebitis, infusion catheter occlusion, and central propagation of clot. If such repeated but temporary central venous catheterization could be conducted by non-physician personnel, such as at a chemotherapy/oncology or hemodialysis outpatient clinic, with a high probability of successful venous access and low risk of complications, such an improvement would make such venous therapy clinically successful.

Embodiments described herein include a subcutaneous needle conduit that attaches to the external adventitial layer of larger veins, arteries, or other biological boundary structures (as discussed below). The subcutaneous conduit can be easily located beneath the skin surface using, for example, tactile sensation, magnetism, metal detection, detection of a signal emitted from a minute transponder, detection of light emission (such as from fluorescent excitation or induced by heat), or through other detection methods.

After a user (e.g., a nurse, technician, or other medical practitioner) locates the subcutaneous conduit, the user may prepare and drape the skin area over the subcutaneous conduit's entry location in a sterile fashion. The user can then advance a sheathed metal needle through the skin and into the subcutaneous conduit. In certain embodiments, the inner lining of the subcutaneous conduit is adapted to prevent or limit perforation by the sharp needle tip, such as by incorporation of perforation resistant material such as Kevlar®, another ballistic plastic, metal, or an appropriate composite material that provides armoring. The user may apply suction to the needle as it is advanced until back flow of blood through the needle is realized. At this point, the user advances the plastic sheath (e.g., composed of PVA, nylon, polyethylene, PVC, polyurethane, or the like, with or without braiding) off of the needle and into the more central venous circulation where it acts, for example, as a chemotherapy infusion catheter or a catheter for the withdrawal or reinjection of blood for hemodialysis. Once the drug infusion or hemodialysis session is completed, the user may remove the catheter. Local manual pressure may be applied to the entry site to ensure minimal bleeding from the zone of venous puncture.

Reference is now made to the figures in which like reference numerals refer to like elements. For clarity, the first digit of a reference numeral indicates the figure number in which the corresponding element is first used. In the following description, numerous specific details are set forth in order to provide a thorough understanding of the present disclosure. However, persons skilled in the art will recognize that certain embodiments can be practiced without one or more of the specific details or with certain alternative equivalent components, materials, and/or methods to those described herein. In other instances, well-known components and methods have not been described in detail so as not to unnecessarily obscure aspects of the present disclosure. Furthermore, the described features, structures, or characteristics may be combined in any suitable manner in one or more embodiments.

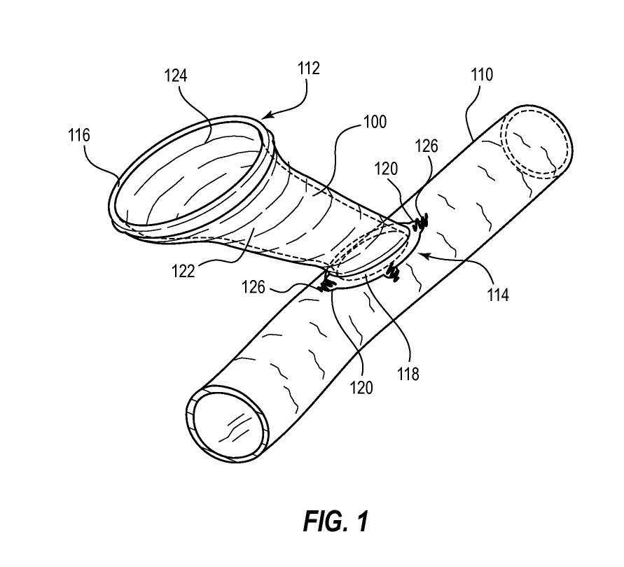

FIG. 1 is a perspective view of a funnel-shaped subcutaneous conduit 100 shown as affixed to a blood vessel 110 according to one embodiment. The funnel-shaped subcutaneous conduit 100 includes a proximal end 112 and a distal end 114. The proximal end 112 includes a proximal ring 116 reinforced with a rigid material such as titanium, Nitinol®, stainless steel, or other material that maintains a desired shape and size of an entry location into the funnel-shaped subcutaneous conduit 100. The circumference of the proximal ring 116 may be selected to provide quick detection below a patient's skin and to provide a sufficient target area for passing a sheathed needle through the patient's skin for access to the blood vessel 110 through the funnel-shaped subcutaneous conduit 100. In one embodiment, a diameter of the proximal ring 116 is in a range between about 1 centimeter (cm) and about 3 cm. However, larger or smaller diameters may also be used. For example, in another embodiment, the diameter of the proximal ring 116 is about 4 cm. The proximal ring 116 also provides increased stiffness to aid in the location of the proximal end 112 of the funnel-shaped subcutaneous conduit 100 by a user using tactile sensation.

The funnel-shaped subcutaneous conduit 100 tapers down from the proximal end 112 to the distal end 114 to guide a needle to a target location at the blood vessel 110. In certain embodiments, an opening 118 in the distal end 114 has a diameter selected to be about the diameter of the particular blood vessel 110 targeted for access. For example, in one embodiment, a diameter of the distal end 114 of the funnel-shaped subcutaneous conduit 100 is in a range between about 8 millimeters (mm) and about 20 mm. The diameter of the opening 118 in the distal end 114 may be larger or smaller than this range. For example, depending on the size of the targeted blood vessel, the diameter of the diameter 118 of the distal end 114 may be as large as 30 mm or 40 mm. Further, in certain embodiments, the opening 118 in the distal end 114 may be larger than the diameter of the particular blood vessel 110 target for access.

As shown in FIG. 1, the distal end 114 may have two, three, four or more tabs 120 (three shown) of material spaced around the opening 118 of the distal end 114. The tabs 120 can serve as anchoring points for attaching the funnel-shaped subcutaneous conduit 100 to the blood vessel 110 or other biological boundary structure. In FIG. 1, the tabs 120 are shown with sutures or helical wires 124 (discussed below) used to affix the funnel-shaped subcutaneous conduit 100 to the adventitial layer of the blood vessel 110.

An external surface 122 of the funnel-shaped subcutaneous conduit 100 may, for example, include an adhesion resistant plastic (such as PTFE), a hydrophilic surface layer, an animal-derived material such as pericardium, or the like. The funnel-shaped subcutaneous conduit 100 may also include an inner lining 124 of, for example, a ballistic plastic, metal or similar material to prevent or limit perforation by an entry needle. Additional shape and/or body may be imparted to the funnel-shaped subcutaneous conduit 100 and the proximal ring 116, according to certain embodiments, by incorporating a layer of hydrogel within the needle-conducting portion (e.g., within the inner lining 124 of the funnel-shaped subcutaneous conduit 100) that swells with the absorption of adjacent water once the funnel-shaped subcutaneous conduit 100 has been deployed or implanted within a patient.

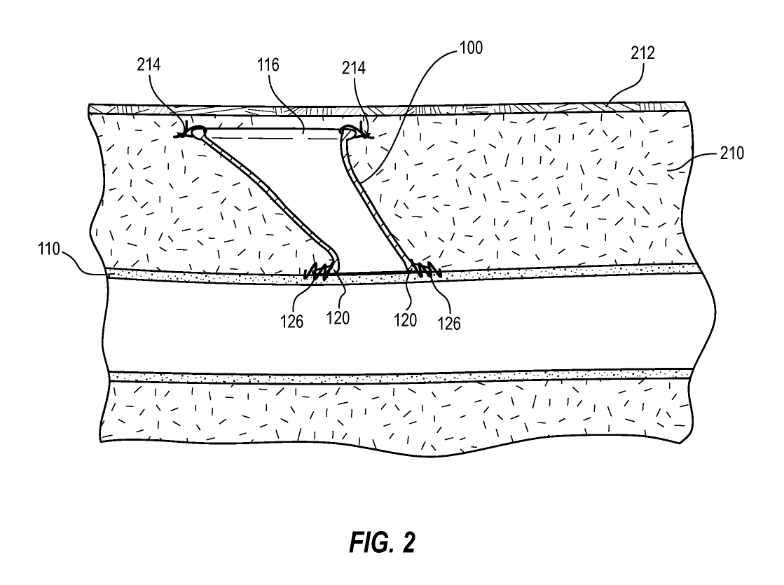

FIG. 2 is a cross-section view of the funnel-shaped subcutaneous conduit 100 shown in FIG. 1 implanted in a patient according to one embodiment. The funnel-shaped subcutaneous conduit 100 may be sized and configured for implantation within subcutaneous tissues 210 so as to provide a needle passageway from a location near the patient's skin 212 to the blood vessel 110. As shown in FIG. 2, helical wires 126 may be used to attach the tabs 120 to the external adventitial layer of the blood vessel 110. As further shown in FIG. 2, certain embodiments may also include one or more sutures 214 placed near the proximal ring 116 or at other locations on the funnel-shaped subcutaneous conduit 100. For example, the funnel-shaped subcutaneous conduit 100 may include a plurality of minute eyelets (not shown) around the periphery of the proximal ring 116 that allow the passage of the sutures 214 to secure the funnel-shaped subcutaneous conduit 100 to the subcutaneous tissues 210.

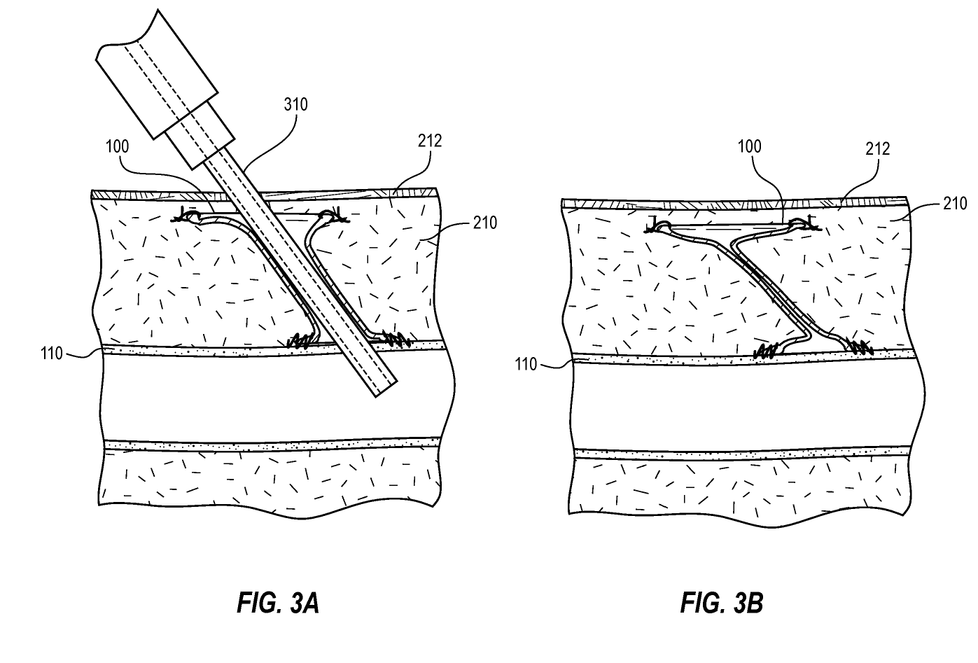

In certain embodiments, the funnel-shaped subcutaneous conduit 100 may be configured for coaptation (reversible collapse) of the walls of the funnel-shaped subcutaneous conduit 100, when not separated by a needle or catheter, to reduce or eliminate central dead space within the funnel-shaped subcutaneous conduit 100. For example, FIG. 3A is a cross-section view of a sheathed needle 310 accessing the blood vessel 110 through the funnel-shaped subcutaneous conduit 100 according to one embodiment. Advancing the sheathed needle 310 through the funnel-shaped subcutaneous conduit 100 forces separation between the walls of the funnel-shaped subcutaneous conduit 100. When the sheathed needle 310 is removed, however, the lack of a minor opening force on the walls allows the funnel-shaped subcutaneous conduit 100 to coapt. FIG. 3B is a cross-section of the funnel-shaped subcutaneous conduit 100 in a coapted state according to one embodiment.

In some embodiments, an optional supporting structure (not shown) of the funnel-shaped subcutaneous conduit 100 may, for example, be provided by a stent-like structure composed of a material such as Nitinol®, Conichrome® (or other chromium-nickel-molybdenum-iron alloy specified by ASTM F1058 or ISO 5832-7), or other elastic or superelastic material. As discussed above, the stent may keep the walls of the body of the funnel-shaped subcutaneous conduit 100 coapted to reduce or obliterate dead space within the needle conduit when not engaged by a needle or catheter. In other embodiments, the elastic or superelastic material may be used to facilitate insertion of the funnel-shaped subcutaneous conduit 100 through minimally invasive surgical tools. For example, the funnel-shaped subcutaneous conduit 100 may be compressed and retained by a sheath during insertion. After insertion, the restraining sheath may be removed to allow the funnel-shaped subcutaneous conduit 100 to expand.

In certain embodiments, magnetic elements or wires (not shown) may also be incorporated into the funnel-shaped subcutaneous conduit 100 to help guide a needle through the funnel-shaped subcutaneous conduit 100 by magnetic deflection. Alternatively, a wire structure (such as a cone composed of woven wires) (not shown) that displays magnetism may be used so that a needle may be guided to a correct vessel puncture point by magnetic deflection. In other words, the magnetic deflection of the wire structure keeps the needle on course toward the correct puncture point. The wire structure may be located in the subcutaneous tissues and attached to the adventitia of a target blood vessel, similar to the funnel-shaped subcutaneous conduit 100 discussed above. The magnetic elements in the guide (and as necessary in the needle) can be as those described in U.S. Pat. No. 7,059,368 issued to Filler (the '368 patent), which is hereby incorporated by reference herein in its entirety. Care is taken to select materials suitable for integration in a mammalian body, as opposed to materials use with vials and the like contemplated in the '368 patent. In magnetically guided embodiments, the armoring can be reduced or dropped, as the magnetic feature guides the needle through the funnel-shaped subcutaneous conduit 100. The needle guide may be substantially a metallic wire frame.

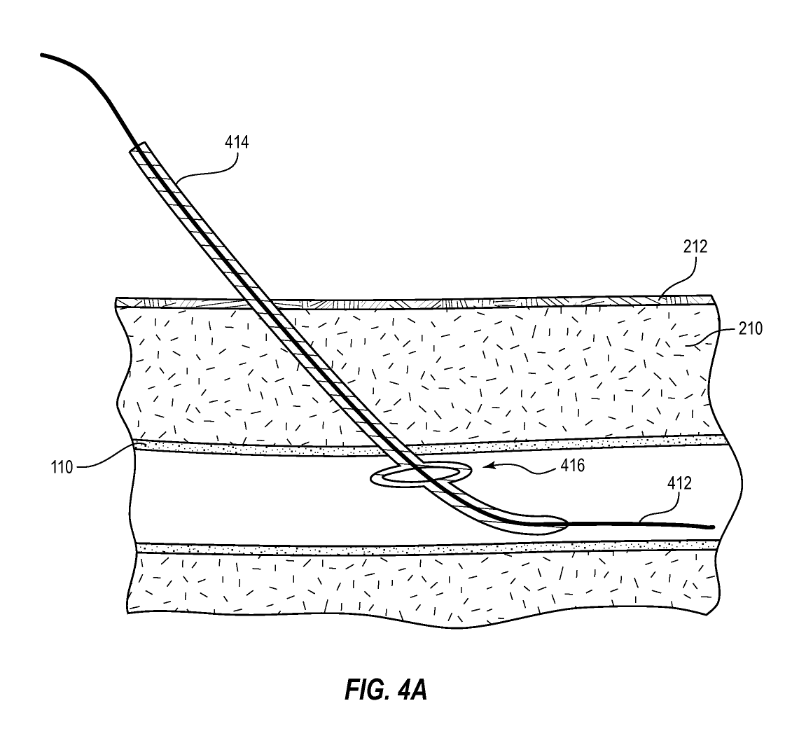

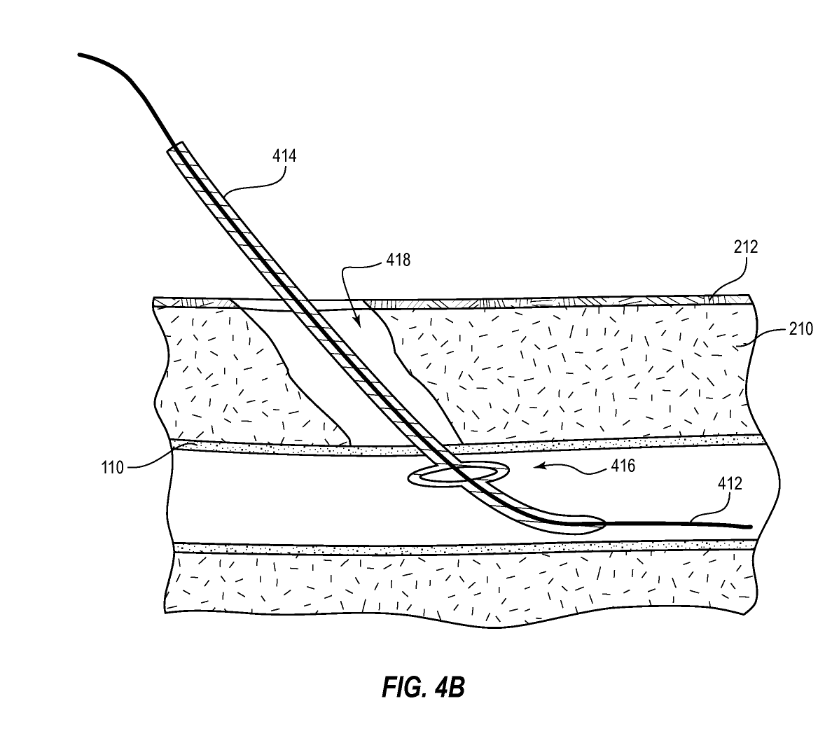

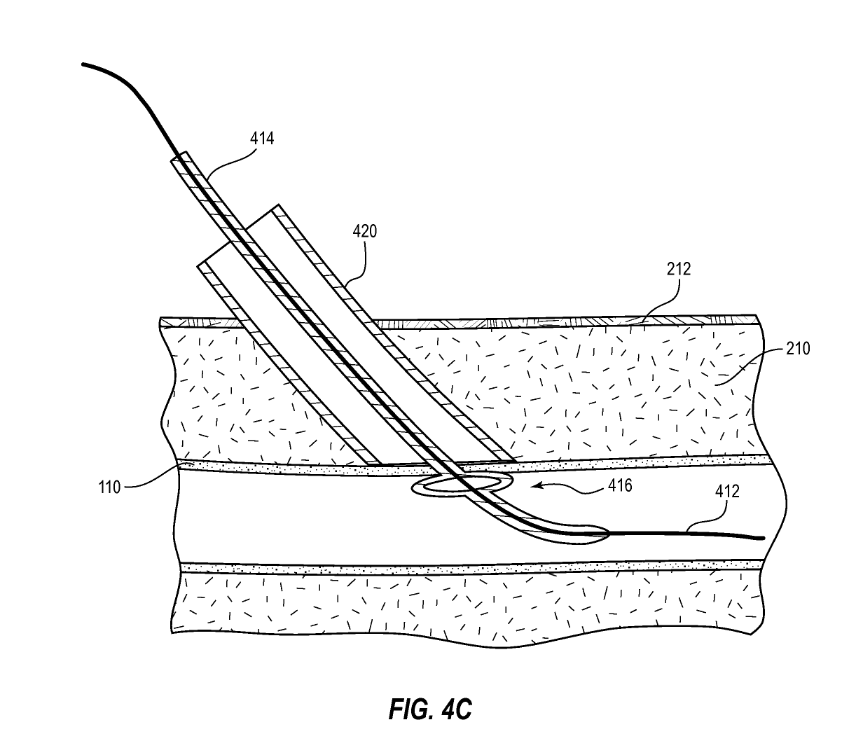

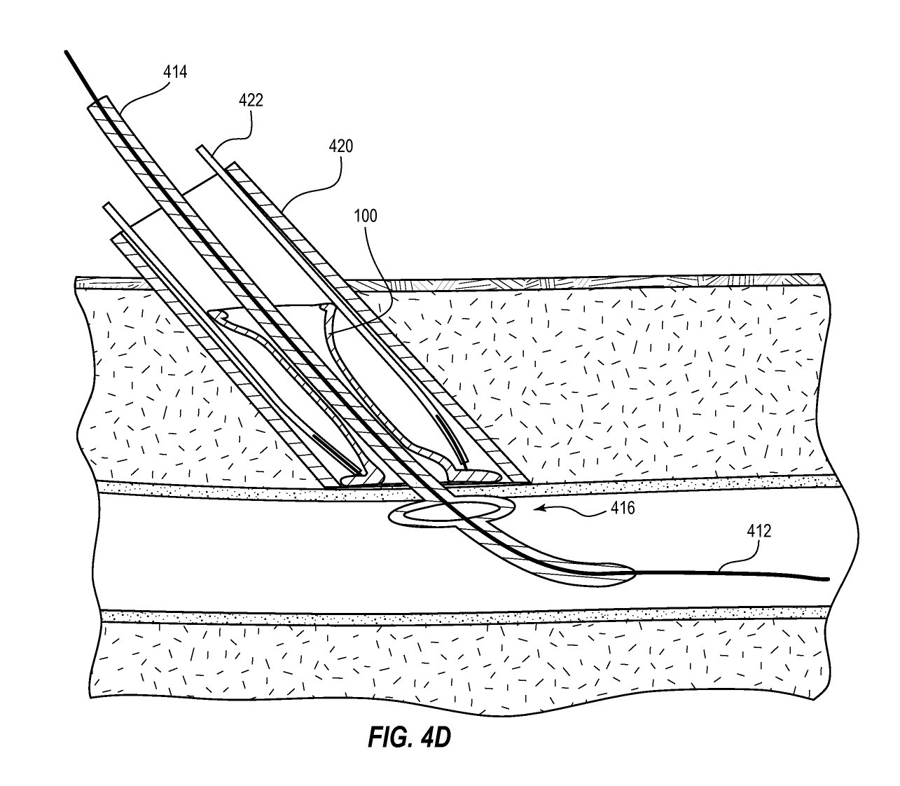

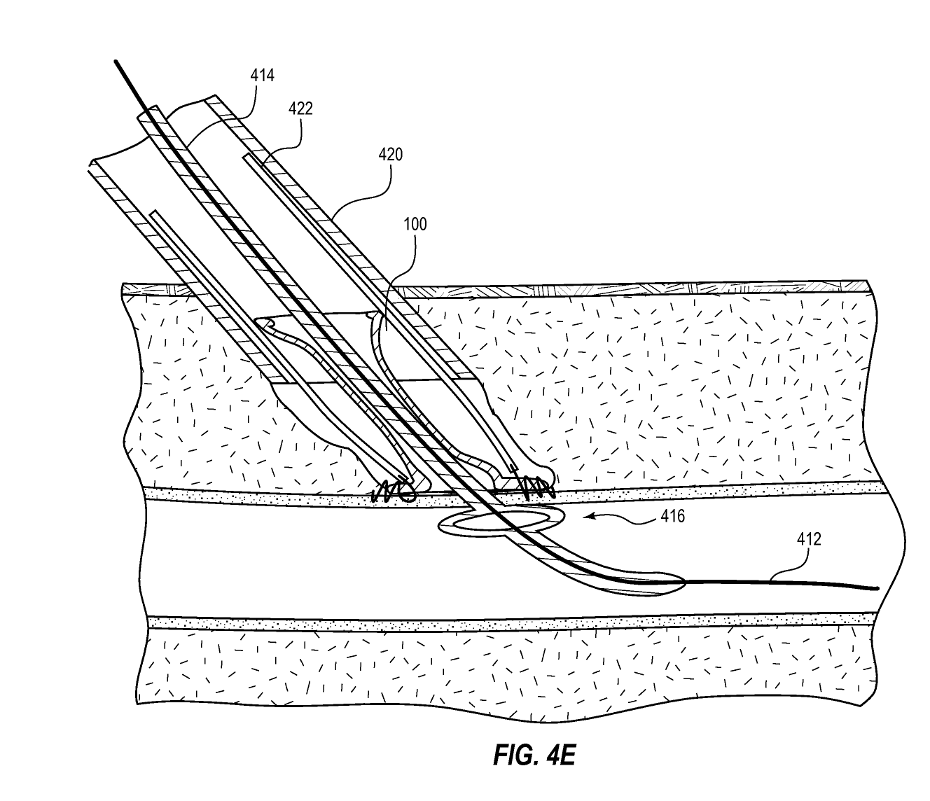

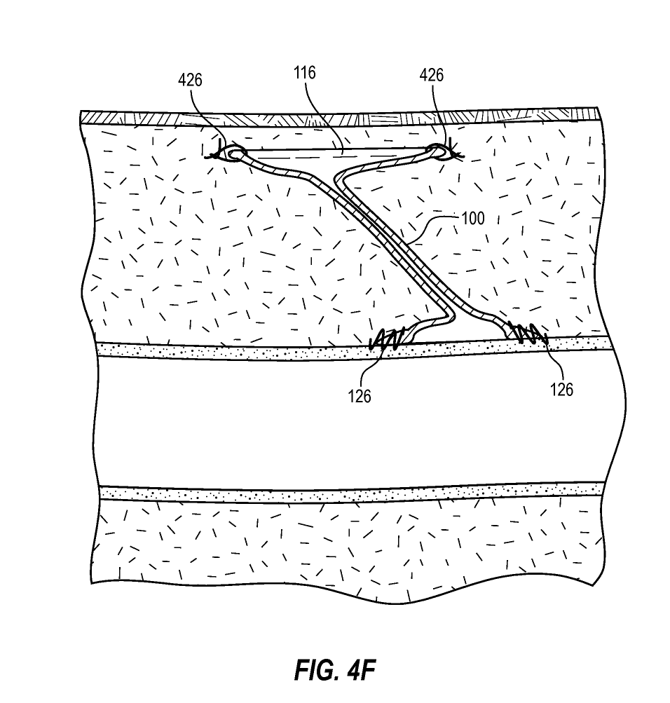

FIGS. 4A, 4B, 4C, 4D, 4E, and 4F are cross-section views illustrating a method for inserting the funnel-shaped subcutaneous conduit 100 into a patient according to one embodiment.

Referring to FIG. 4A, the blood vessel 110 (e.g., any target vein or artery) may first be punctured with a micropuncture (not shown) (e.g., a 21 gauge needle+0.018″ wire+4-5 F entry sheath) system. This allows placement of a guidewire 412 (e.g., a stiff 0.035″ guidewire) over which a dilator 414 (e.g., 6-8 F) with a retaining segment 416 (e.g., a Malecot segment with two to four “wings”) may be advanced into the blood vessel 110. The retaining segment 416 may be extended/deployed such that when the dilator 414 is pulled back it is stopped at the vessel entry point by the retaining structure 416.

Referring to FIG. 4B, the skin entry zone of the dilator 414 may be incised with a scalpel and a subcutaneous pouch 418 for the funnel-shaped subcutaneous conduit 100 may be bluntly dissected.

Referring to FIG. 4C, over the dilator 414, a sheath 420 (e.g., a 10-12 F beveled tip sheath) with its dilator (not shown) may be advanced to the adventitial surface of the target blood vessel 110. The sheath's dilator may then be removed.

Referring to FIG. 4D, through the sheath 420, the operator may advance the funnel-shaped subcutaneous conduit 100 (which may be in a compressed state). As discussed above, the distal end 114 end of the funnel-shaped subcutaneous conduit 100 may include tabs 120 (e.g., two or more) adapted to facilitate attachment of the distal end 114 to the adventitial layer of the blood vessel 110. Hypotubes 422, one attached to each tab 120, may be advanced down the sheath 420 with the funnel-shaped subcutaneous conduit 100. Each hypotube 422 includes therein a minute “curly-Q” or helically shaped length of wire (referred to herein as helical wire 126) (e.g., of superelastic Nitinol® or other suitable material) with a sharp distal end. A portion of each helical wire 126 is adapted to attach to a respective tab 120 at the distal end 114 of the funnel-shaped subcutaneous conduit 100.

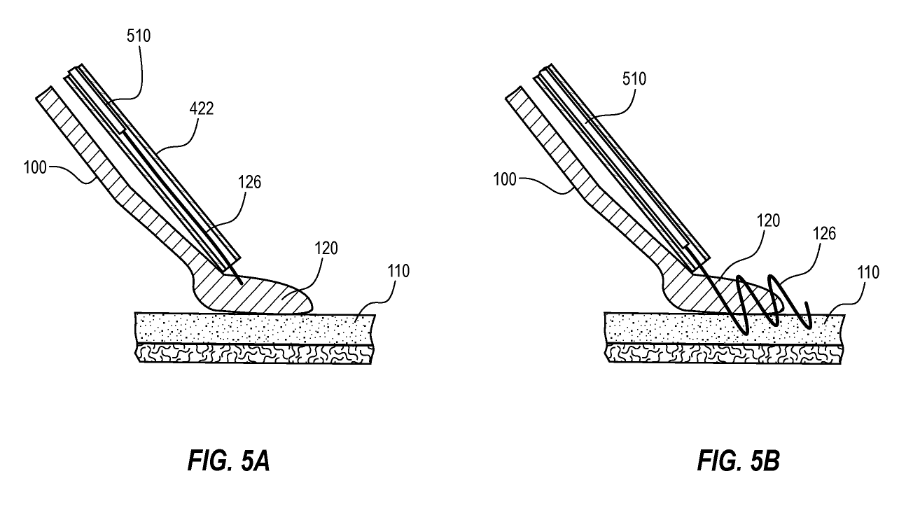

For example, FIGS. 5A and 5B are enlarged cross-section views of a hypotube 422 positioned adjacent to a tab 120 of the funnel-shaped subcutaneous conduit 100 according to one embodiment. As shown in FIG. 5A, while in the hypotube 22, the helical wire 126 may be in a straight configuration. In one embodiment, the helical wire 126 comprises superelastic Nitinol® that is restrained by the hypotube 422 so as to be in the straight configuration. As shown in FIG. 5A, the sharp distal end of the helical wire 126 may extend from the distal end of the hypotube 422 so as to attach to the tab 120. The operator may then depress a proximal plunger 510 to force the helical wire 126 from the distal end of the hypotube 422. As shown in FIG. 5B, when the wire 126 is advanced out of the distal end of the hypotube by depressing the proximal plunger 510 on the hypotube 422, the superelastic wire 126 begins to reassume its predetermined helical shape. As the helical wire 126 is advanced out of the hypotube 422, the sharp distal end of the of the helical wire 126 repeatedly drives the helical wire 126 in and back out of the tab 120 and the adventitial layer of the blood vessel 110.

Referring to FIG. 4E, the operator knows when the hypotubes 422 are in the correct location for ligating wire deployment by advancing the hypotubes 422 within the sheath 420 until resistance is felt against the retaining segments 416 within the vessel lumen. As illustrated, before deploying the ligating mechanism (e.g., before depressing the proximal plungers 210 to force the helical wires 126 out the distal ends of the hypotubes 422), the sheath 420 may be withdrawn slightly as needed to facilitate the ligating mechanism. After wire deployment (which attaches the distal conduit tabs 120 to the external surface of the blood vessel 110), the hypotubes 422 can be removed. A pusher device (not shown), located against the collapsed proximal ring structure, is held stationary while the sheath 21 and hypotubes 422 are withdrawn, leaving the funnel-shaped subcutaneous conduit 100 (in a collapsed state in the illustrated embodiment) within the perivascular and subcutaneous tissues 210. Any exposed portion of the proximal end 112 of the funnel-shaped subcutaneous conduit 100 can be “tucked” into the subcutaneous tissue pouch 418. In certain embodiments, one or several minute eyelets (not shown) may be present along the periphery of the proximal ring 116 or at other locations on the funnel-shaped subcutaneous conduit 100, allowing the funnel-shaped subcutaneous conduit 100 to be sutured to the subcutaneous tissues 210 within the pouch 418. The retaining segment 416 of the dilator 414 may be retracted or collapsed, and the dilator 414 removed. Direct manual pressure may be applied to the entry site for several minutes to ensure hemostasis. Finally, the entry site incision may be repaired with subcuticular sutures (not shown). The funnel-shaped subcutaneous conduit 100 should be ready for use within ten to fourteen days.

FIG. 4F shows the funnel-shaped subcutaneous conduit 100 with sutures 426 securing the proximal ring 116 to the subcutaneous tissue 210. The entry site incision is shown as repaired and the funnel-shaped subcutaneous conduit 100 is shown as ready for use. The funnel-shaped subcutaneous conduit 100 may be used for repetitive entry into both veins and arteries. However, the funnel-shaped subcutaneous conduit 100 may also be useful for repetitive entry into other bodily cavities, such as the cerebrospinal fluid (CSF) space, the biliary tree, the urinary system, etc. The funnel-shaped subcutaneous conduit 100 may also serve as a pathway to allow rapid reentry for deep body access for biopsies, fluid drainage, laparoscopy, etc. The funnel-shaped subcutaneous conduit 100 may also allow repetitive access to the inferior vena cava and the abdominal aorta via the trans lumbar route. In these cases, initial access of the target vessel or other structures may, for example, be accomplished under computed tomography (CT) scan guidance (or other imaging guidance) with skinny needle puncture. The structures against which the funnel-shaped subcutaneous conduit 100 may be affixed to facilitate periodic piercing can be termed “biological boundary structures.”

The above description of implanting the funnel-shaped subcutaneous conduit 100 within a patient has been described with regards to an embodiment wherein the funnel-shaped subcutaneous conduit 100 remains coapted until a sheathed needle is inserted therethrough to access the blood vessel 110. In other embodiments, however, the funnel-shaped subcutaneous conduit 100 may be inserted in a collapsed state, but may then be expanded in place to create an open passageway to the blood vessel 110. For example, the sheath 420 may be configured to restrain the funnel-shaped subcutaneous conduit 100 in a collapsed state during insertion, and removal of the sheath 420 allows the funnel-shaped subcutaneous conduit 100 to expand within the perivascular and subcutaneous tissues 210. In another embodiment, a balloon (not shown) may be inflated to expand the funnel-shaped subcutaneous conduit 100 after insertion.

An artisan will recognize from the disclosure herein many alternatives for implanting the funnel-shaped subcutaneous conduit 100 within a patient. For example, a system for remotely ligating the funnel-shaped subcutaneous conduit 100 to the adventitial layer of the vessel 110 may be similar to the remote ligation system marketed as the Q-wire by the Davol division of C.R. Bard Inc. Other remote ligation systems that may be used with the funnel-shaped subcutaneous conduit 100 include crimping of a metallic or resorbable surgical clip, which may be remotely engaged with the blood vessel 110 for example by pulling back on a plunger in the deploying device. A resorbable or nonresorbable surgical suture may also be used to affix the funnel-shaped subcutaneous conduit 100 to the target vessel 110.

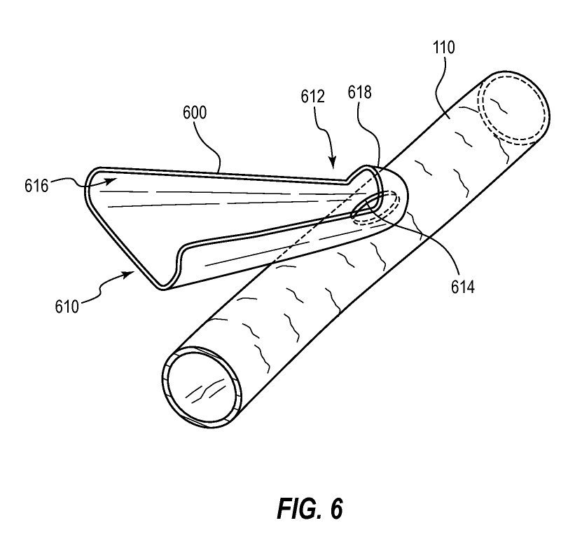

Further, needle conduits having other shapes (rather than the illustrated funnel shape) may also be used. For example, FIG. 6 is a perspective view of a sluice-shaped or “dust pan” subcutaneous conduit 600 according to one embodiment. The sluice-shaped subcutaneous conduit 600 is shown situated on a blood vessel 110. The sluice-shaped subcutaneous conduit 600 includes a first end 610 that is wider than a second end 612. The wider first end 610 serves to locate a needle entry point and provides guidance for directing the needle to the second end 612. The second end 612 includes an eyelet 614 that is shown positioned over an intended venous entry point. The sluice-shaped subcutaneous conduit 600 includes folded over lips 616 at the edges of the sluice-shaped subcutaneous conduit 600 that help direct a needle towards the eyelet 614 affixed to the blood vessel 110. As shown, the sluice-shaped subcutaneous conduit 600 may include a hood 618 over the eyelet 614 to prevent the needle from sliding beyond the intended venous entry point and to further direct the needle to the eyelet 614. Although not shown, second end 612 of the sluice-shaped subcutaneous conduit 600 may include tabs for suturing the second end 612 to the adventitial layer of the blood vessel 110.

Other systems and methods may also be used to implant a subcutaneous conduit within a patient. For example, in one embodiment, two guidewires are used. In such an embodiment, a first guidewire is advanced through a first incision directly into a target vessel. A second guidewire is inserted through a second incision and tunneled under the skin to the first incision location similar to, for example, the tunneling of a Hickman catheter. The second guidewire is used for insertion of the subcutaneous conduit. In some embodiments, the subcutaneous conduit may be in a collapsed state during insertion and may be expanded in place by, for example, removing a sheath or expanding a balloon. Once the subcutaneous conduit is in place, the first guidewire is used for affixing the eyelet of the subcutaneous conduit directly to a desired location of the target vessel. For example, the first guidewire may be used with a suture device (such as a Perclose® suture device or other mechanical closure device) to precisely place sutures at or near the eyelet or distal opening of the subcutaneous conduit. Both guidewires may then be removed and both incisions repaired. The subcutaneous conduit may then be used after healing for ten to fourteen days.

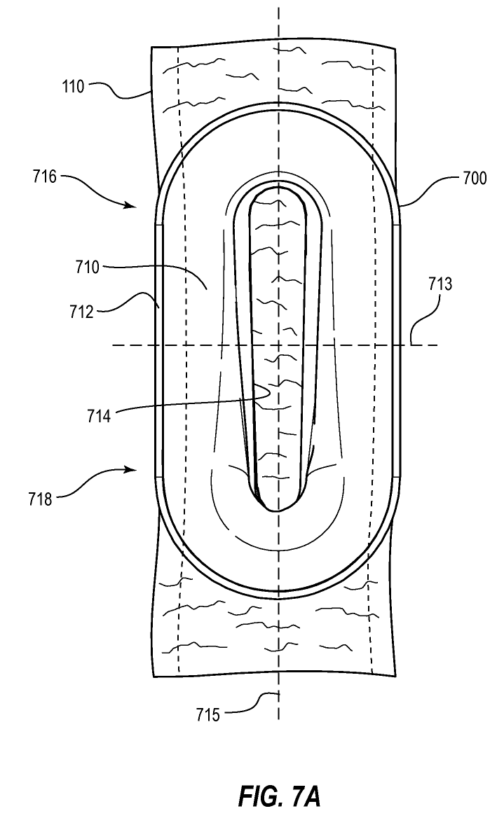

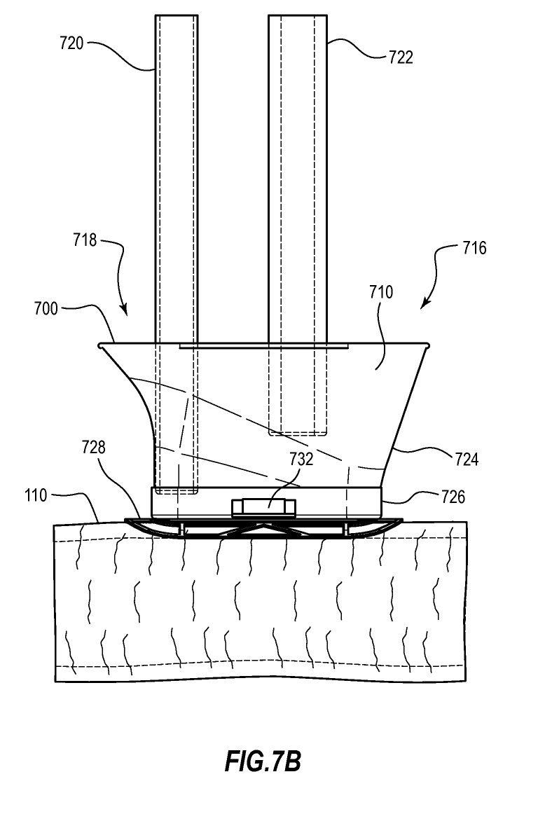

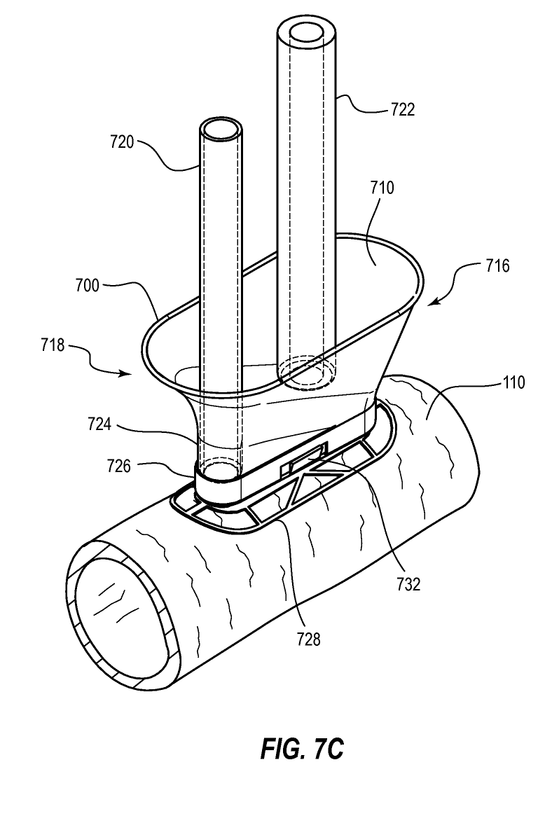

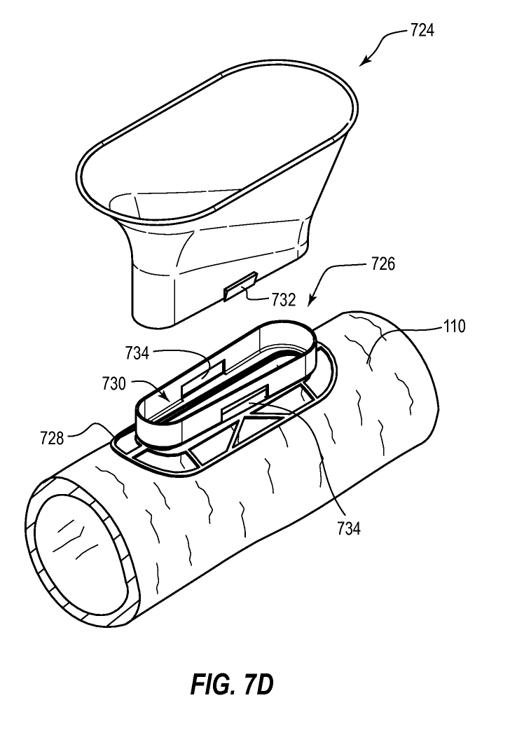

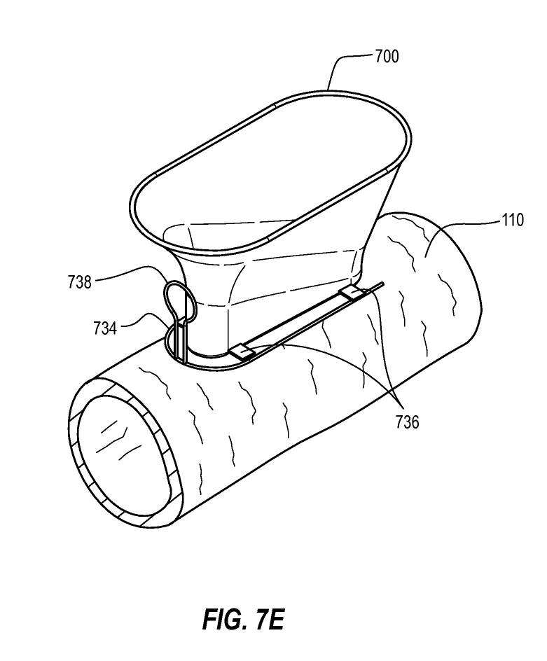

FIGS. 7A, 7B, 7C, 7D, and 7E schematically illustrate an elongated funnel-shaped subcutaneous conduit 700 according to certain embodiments. The elongated funnel-shaped subcutaneous conduit 700 is shown with respect to a blood vessel 110. Repeated access to the same site can result in damage to the blood vessel 110. Thus, a user may access different locations along the blood vessel 110 through the elongated funnel-shaped subcutaneous conduit 700 by selecting different sizes of canulas. On one day, for example, a user may access the blood vessel 110 at one end of the elongated funnel-shaped subcutaneous conduit 700 by using a canula with a relatively small outer diameter. On another day, the user may access the blood vessel 110 at another end of the elongated funnel-shaped subcutaneous conduit 700 by using a canula with a relatively large outer diameter.

FIG. 7A illustrates a top view of the elongated funnel-shaped subcutaneous conduit 700, as attached to the blood vessel 110. The elongated funnel-shaped subcutaneous conduit 700 includes walls 710 that narrow from a proximal opening 712 to a distal opening 714. The proximal opening 712 and the distal opening 714 each have a short axis 713 and a long axis 715. The elongated funnel-shaped subcutaneous conduit 700 is implanted in a patient such that the distal opening 714 is adjacent the blood vessel 110 with the long axis 715 aligned with the axis of the blood vessel 110. Thus, after the oval funnel-shaped subcutaneous conduit 700 is implanted, a user locates the large proximal opening 712 and inserts a sheathed needle or other canula (see FIGS. 7B and 7C), which the walls 710 of the funnel-shaped subcutaneous conduit 700 directs to the blood vessel 110 through the distal opening 714.

The short axis 713 of the distal opening 714 is larger at a first end 716 than it is at a second end 718 of the elongated funnel-shaped subcutaneous conduit 700. In other words, the distal opening 714 tapers in size from the first end 716 to the second end 718. The taper directs a canula with a relatively larger outer diameter to the larger end 716 of the elongated funnel-shaped subcutaneous conduit 700, while allowing a canula with a smaller outer diameter to pass through the smaller end of the of the distal opening 714. The tapering allows a user to select one of several access points along the vessel 110 by selecting the diameter of the access canula.

FIG. 7B illustrates a side view of the elongated funnel-shaped subcutaneous conduit 700, as attached to the blood vessel 110. FIG. 7C illustrates a perspective view of the elongated funnel-shaped subcutaneous conduit 700, as attached to the blood vessel 110. For illustrative purposes, FIGS. 7B and 7C show a first canula 720 and a second canula 722 that a user may select for gaining access to the blood vessel 110. A user may, for example, decide to use the first canula 720 at a first time to access the blood vessel 110 at a first site and the second canula 722 at a second time to access the blood vessel 110 at a second site. As shown, the first canula 720 has an outer diameter that is smaller than the outer diameter of the second canula 722. Thus, the first canula 720 may access the blood vessel at the narrower or second end 718 of the elongated funnel-shaped subcutaneous conduit 700, whereas second canula 722 is sufficiently large such that it can access the blood vessel 110 at only the wider or first end 716 of the elongated funnel-shaped subcutaneous conduit 700. Although not shown, additional canulas having different outer diameters may be used to access different sites along the length of the elongated funnel-shaped subcutaneous conduit 700 at desired points between the first end 716 and the second end 718. The sides 710 of the elongated funnel-shaped subcutaneous conduit 700 may also be sloped towards the larger end 716 of the distal opening 714 (shown in FIG. 7A) to further direct larger canula 722 to the larger end 716 of the elongated funnel-shaped subcutaneous conduit 700.

The embodiment shown in FIGS. 7B, 7C, and 7D is a two-piece design that includes a funnel portion 724 and a base portion 726. FIG. 7D is a perspective view illustrating the funnel portion 724 separated from the base portion 726. In the illustrated embodiment, the base portion 726 includes a curved, flexible lattice 728 configured for suturing to the wall (e.g., the adventitial layer) of the blood vessel 110. For illustrative purposes, the drawings do not show the sutures securing the base portion 726 to the blood vessel 110. As shown in FIG. 7D, the base portion may include a pocket 730 for receiving the funnel portion 724. The funnel portion 724 may snap into the base portion 726. For example, as illustrated, the funnel portion 724 may include two retaining tabs 732 (one on either side) protruding from the funnel portion 724 that fit into matching openings 734 in the base portion 726. Squeezing the sides of the funnel portion 724 releases the retaining tabs 732 from the openings 734 to separate the funnel portion 724 from the base portion 726. The two-piece design provides easier access to the base portion 726 during the implant and explant procedures. For example, a user may implant the elongated funnel-shaped subcutaneous conduit 700 by excising a pocket of subcutaneous tissue (not shown) sufficient to fit the elongated funnel-shaped subcutaneous conduit 700, suturing the base portion 726 to the adventitial layer of the blood vessel 110 (e.g., using a suturing system), and snapping the funnel portion 724 into place in the base portion 726. To explant the elongated funnel-shaped subcutaneous conduit 700, a user may squeeze the sides of the funnel portion 724 to release the funnel portion 724 from the base portion 726 so that the sutures may be cut and removed.

FIG. 7E is a perspective view of a one-piece funnel design (e.g., that does not include the base portion 726 illustrated in FIGS. 7B, 7C, and 7D) of the elongated funnel-shaped subcutaneous conduit 700, as attached to the blood vessel 110. The embodiment of the elongated funnel-shaped subcutaneous conduit 700 shown in FIG. 7E may be attached to a suture wire 734 through a plurality of tabs 736 (two shown). The suture wire 734 may be located along both sides of the elongated funnel-shaped subcutaneous conduit 700. Sutures (not shown) may be used to secure the suture wire 734 to the adventitial layer of the blood vessel 110. Thus, the attached elongated funnel-shaped subcutaneous conduit 700 is secured to the blood vessel 110. The suture wire 734 may include a central tab 738 that may be pulled like a rip-cord to release the elongated funnel-shaped subcutaneous conduit 700 from the blood vessel 110. When the central tab 738 is pulled, the suture wire 734 separates from the tabs 736 such that the suture wire 734 may slip free from the sutures, which also allows the elongated funnel-shaped subcutaneous conduit 700 to be removed from the patient. Thus, a user may easily access and remove the sutures.

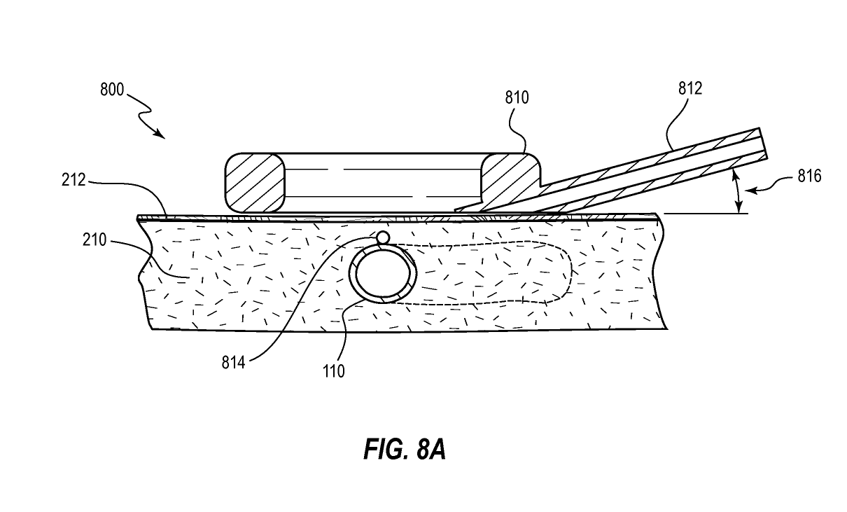

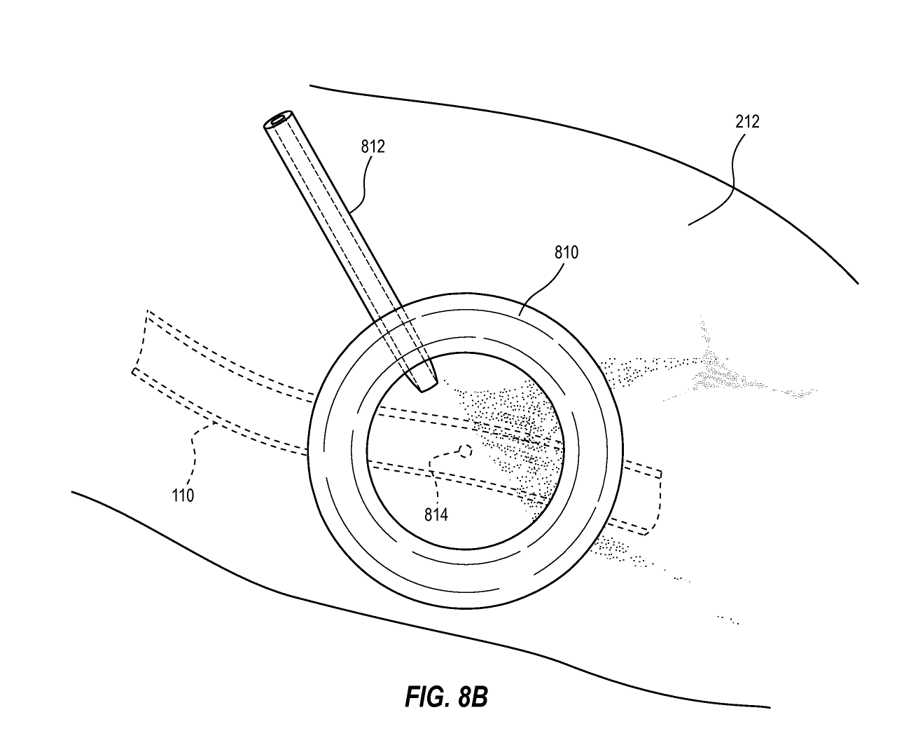

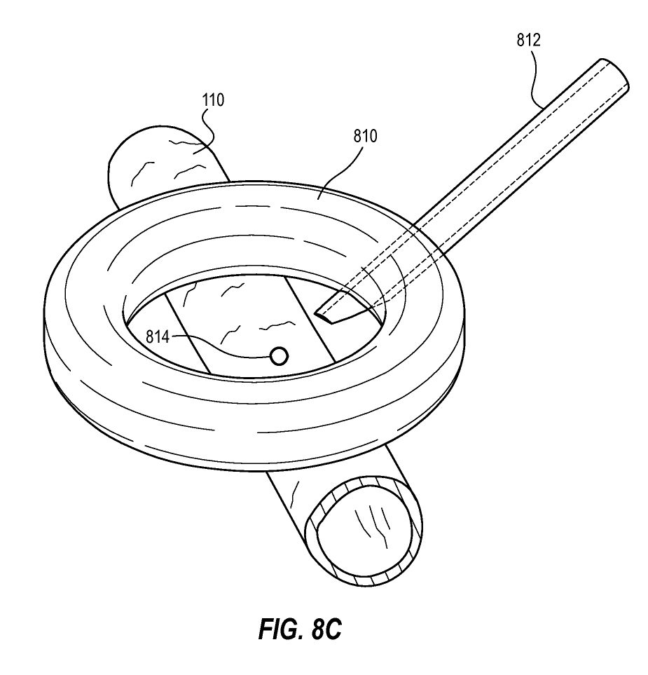

FIGS. 8A, 8B, and 8C schematically illustrate a metal detection system 800 for accessing a blood vessel 110 according to one embodiment. FIG. 8A is a cross-section side view of the metal detection system 800 in use external to a patient's skin 212 to locate a desired entry site on the blood vessel 110 located in subcutaneous tissue 210. FIG. 8B is a top view of the metal detection system 800 in use and FIG. 8C is a perspective view of the metal detection system 800 in use (with the skin 212 and subcutaneous tissue 210 omitted for illustrative purposes).

The metal detection system 800 includes one or more metal detector coils (not shown) within a coil housing 810 attached to a guide canula 812. The metal detection system 800 also includes a metallic implant 814. Although not shown, the metal detection system 800 also includes an oscillator for producing an alternating current that passes through the one or more coils to produce an alternating magnetic field. When the coil housing 810 is sufficiently close the metallic implant 814, the alternating magnetic field generated by the magnetic coils produce eddy currents in the metallic implant 814 such that the metallic implant 814 produces another alternating magnetic field. The one or more coils are then used to detect the alternating magnetic field produced by the metallic implant 814. The metal detection system 800 may provide audio and/or visual indicia of metal detection.

Thus, the metal detection system 800 may be used to detect the metallic implant 814. The guide canula 812 may be rigidly fixed to the coil housing 810 to provide accurate guidance of an access needle to the desired access site near the detected metallic implant 814.

In one embodiment, the metallic implant 814 comprises a small spherical-shaped piece of metal suitable for human implant, such as stainless steel or titanium. The metallic implant 814 may be placed on or above the targeted blood vessel 110, for example, using open surgery or with a low invasive procedure through a small hypodermic needle. In certain embodiments, the metallic implant 814 may be part of or integrated with a subcutaneous conduit, such as the subcutaneous conduit embodiments described herein. Given fixed properties (e.g., material and mass) of the metallic implant 814, the metal detector system 800 may be calibrated to accurately find both the planar (e.g., in X and Y directions) position and the depth (e.g., in a Z direction) of the metallic implant 814. Thus, in certain embodiments, an angle 816 of the guide canula 812 with respect to a plane of the patient's skin 212 may be adjusted based on the detected depth of the metallic implant 814. In one such embodiment, the guide canula 812 may include a hinge structure (not shown) to allow the angle 816 to be adjusted based on the detected depth. In another embodiment, a user may select one of multiple fixed guide canulas 812 that each provide a different angle 816 based on the metal detector's depth reading.

In addition, or in other embodiments, a platform (not shown) may be used to steady the metal detector system 800 over the patient to increase accuracy.

It will be understood by those having skill in the art that many changes may be made to the details of the above-described embodiments without departing from the underlying principles of the invention. The scope of the present invention should, therefore, be determined only by the following claims.

1. A subcutaneous conduit for implanting in a patient to allow repeated access to a blood vessel, comprising:
a tapered guide segment for guiding a needle through subcutaneous tissue to the blood vessel;
a proximal opening for receiving the needle into the guide segment; and
a distal opening for passing a tip of the needle out of the guide segment into the blood vessel, the distal opening sized and configured to be attached directly to a surface of the blood vessel,
wherein the proximal opening is wider than the distal opening such that a width of the guide segment tapers from the proximal opening to the distal opening.

2. The subcutaneous conduit of claim 1, further comprising:
a rigid proximal ring forming the proximal opening.

3. The subcutaneous conduit of claim 2, wherein the guide segment comprises eyelets for suturing the rigid proximal ring to the subcutaneous tissue.

4. The subcutaneous conduit of claim 2, wherein the rigid proximal ring is substantially circular.

5. The subcutaneous conduit of claim 2, wherein the guide segment comprises an elastic or superelastic material that allows the subcutaneous conduit to be in a collapsed state during implantation.

6. The subcutaneous conduit of claim 5, wherein the guide segment is configured to transform from the collapsed state to an open state after implantation.

7. The subcutaneous conduit of claim 5, wherein the guide segment is configured to remain in a coapted state after implantation until opened by a force of the needle or another canula being pushed through the guide segment.

8. The subcutaneous conduit of claim 1, wherein the guide segment is armored to resist puncturing thereof by the tip of the needle.

9. The subcutaneous conduit of claim 1, where in the guide segment includes one or more magnetic elements to assist in guiding the tip of the needle to the distal opening.

10. The subcutaneous conduit of claim 1, further comprising:
one or more tabs located near the distal opening for affixing the distal opening to the surface of the blood vessel.

11. The subcutaneous conduit of claim 10, further comprising:
helical wire comprising a superelastic material for affixing the one or more tabs to an adventitial layer of the blood vessel.

12. The subcutaneous conduit of claim 1, wherein a diameter of the distal opening is about equal to a diameter of the blood vessel.

13. The subcutaneous conduit of claim 1, wherein the subcutaneous conduit comprises an elongated funnel shape.

14. The subcutaneous conduit of claim 13, wherein a first axis of the subcutaneous conduit is substantially longer than a second axis of the subcutaneous conduit, the first axis being perpendicular to the second axis, and wherein the distal opening is configured to be attached to the surface of the blood vessel such that the first axis is about parallel to a cylindrical axis of the blood vessel.

15. The subcutaneous conduit of claim 14, wherein a width, in the direction of the second axis, of the distal opening narrows along the length of the first axis such that a first canula comprising a first outer diameter fits through a first end of the distal opening and a second canula comprising a second outer diameter fits through a second end of the distal opening, wherein the second outer diameter does not allow the second canula to fit through the first end of the distal opening.

16. The subcutaneous conduit of claim 15, wherein the tapered guide segment is sloped to guide the second canula from the first end of the distal opening to the second end of the distal opening.

17. The subcutaneous conduit of claim 13, wherein the guide segment comprises retaining tabs located near the distal opening, the subcutaneous conduit further comprising a base portion comprising:
a curved, flexible lattice configured for suturing to the wall of the blood vessel;
a pocket for receiving the distal opening; and
slots for engaging the retaining tabs of the guide segment to thereby attach the distal opening to the surface of the blood vessel.

18. The subcutaneous conduit of claim 13, further comprising:
a plurality of tabs each having a first end attached to the distal opening; and
a suture wire attached to a second end of each of the tabs, the suture wire configured to be sutured to the surface of the blood vessel.

19. A method for repeatedly accessing a blood vessel, comprising:
making an incision in a patient's skin at an incision location;
at the incision location, excising a pouch in subcutaneous tissue for receiving a funnel-shaped conduit including a proximal opening, a distal opening, and a tapered guide segment extending between the proximal opening and the distal opening;
inserting the funnel-shaped conduit into the pouch;
securing the distal opening of the funnel-shaped conduit directly to a surface of the blood vessel;
securing the proximal opening to the subcutaneous tissue;
repairing the incision; and
after a healing period, inserting a needle through the patient's skin so as to enter the guide segment through the proximal opening and advance a tip of the needle through the distal opening into the blood vessel.

20. The method of claim 19, further comprising:
inserting a guidewire through the incision into the blood vessel;
inserting a dilator over the guidewire into the blood vessel, the dilator comprising a retaining segment that is advanced into the blood vessel;
deploying the retaining segment to maintain a distal end of the dilator within the blood vessel;
inserting a sheath over the dilator into the pouch; and
wherein inserting the funnel-shaped conduit comprises inserting the funnel-shaped conduit over the dilator into the sheath.

21. The method of claim 20, wherein the funnel-shaped conduit comprises a tab located near the distal opening, the method further comprising:
attaching a distal end of a superelastic wire to the tab, the superelastic wire being restrained within a hypotube;
inserting the hypotube along with the funnel-shaped conduit into the sheath;
pressing a proximal plunger to force the superelastic wire out a distal end of the hypotube, wherein the superelastic wire reassumes a helical shape upon exiting the hypotube, and wherein forcing the superelastic wire out the distal end of the hypotube cases the distal end of the superelastic wire to repeatedly pass through the tab and an adventitial layer of the blood vessel to secure the distal opening to the distal surface of the blood vessel.

22. A subcutaneous conduit comprising:
an eyelet segment adapted to engage against the surface of a biological boundary structure;
a guide segment, including a puncture-resistant surface shaped to narrow such that a needle can first engage a larger segment and be guided by a shaped, narrowing surface towards the eyelet segment; and
tabs, located near the eyelet segment, adapted to engage a ligating mechanism for attaching the needle conduit to the biological boundary structure.

23. The subcutaneous conduit of claim 22, wherein the guide segment is funnel-shaped.

24. The subcutaneous conduit of claim 22, wherein the guide segment is shaped as an elongated funnel to selectively provide access to a plurality of different access sites along an axis of the biological boundary structure.

25. The subcutaneous conduit of claim 22, wherein the guide segment is sluice-shaped.

26. A subcutaneous conduit comprising:
an eyelet segment adapted to engage against the surface of a biological boundary structure;
a guide segment, including means for guiding the needle towards the eyelet segment; and
means, located near the eyelet segment, adapted to engage a ligating mechanism for attaching the needle conduit to the biological boundary structure.

27. The subcutaneous conduit of claim 26, wherein the guide segment is funnel-shaped.

28. The subcutaneous conduit of claim 26, wherein the guide segment is shaped as an elongated funnel to selectively provide access to a plurality of different access sites along an axis of the biological boundary structure.

29. The subcutaneous conduit of claim 26, wherein the guide segment is sluice-shaped.

30. A system for accessing a biological boundary structure at a desired entry site within a mammalian body, the system comprising:
one or more metal detector coils within a coil housing, the one or more metal detector coils configured to detect a metallic implant within the mammalian body; and
a guide canula attached to the coil housing at an angle such that placement of the one or more metal detector coils over the metallic implant aligns an opening through the guide canula with desired entry site.

31. The system of claim 30, wherein the angle of the guide canula is adjustable based on a detected depth of the metallic implant within the mammalian body.

32. The system of claim 30, wherein the angle of the guide canula is fixed.

33. The system of claim 30, further comprising the metallic implant affixed to a surface of the biological boundary structure within the mammalian body.

34. The system of claim 30, further comprising a subcutaneous conduit comprising the metallic implant, the subcutaneous conduit configured to be affixed directly to a blood vessel.
